(19) United States
(12) Patent Application Publication
(10) Pub. No.: US 2025/0134435 A1
(43) Pub. Date: May 1, 2025

(54) WEARABLE SENSOR HAVING DRY ELECTRODES, AND A MANUFACTURING METHOD

(71) Applicants: André Filipe Marques-Smith, New Castle, DE (US); Ana Montero Horas, New Castle, DE (US); Catia Figueiredo, New Castle, DE (US); Goncalo Narciso, New Castle, DE (US); Joana Neto, New Castle, DE (US)

(72) Inventors: André Filipe Marques-Smith, New Castle, DE (US); Ana Montero Horas, New Castle, DE (US); Catia Figueiredo, New Castle, DE (US); Goncalo Narciso, New Castle, DE (US); Joana Neto, New Castle, DE (US)

(21) Appl. No.: 18/925,640

(22) Filed: Oct. 24, 2024

(30) Foreign Application Priority Data
Oct. 30, 2023 (GB) 2316544.2

(51) Int. Cl. A61B 5/268; A61B 5/291

(52) U.S. Cl. CPC A61B 5/268; A61B 5/291; A61B 2562/0209; A61B 2562/125

(57) ABSTRACT

A skin-conformable and compact wearable sensor for monitoring surface physiological and/or surface brain signals of the wearer. The wearable sensor comprises dry electrodes formed from an electrically-conductive polymer composition, the electrically-conductive polymer composition comprising: an electrically-conductive material comprising particulate carbon; at least one additive; and a silicone polymer. The particulate carbon may be present in a range from 5 wt. % to 20 wt. % by weight of the electrically-conductive polymer composition.

### CROSS-REFERENCES TO RELATED APPLICATIONS

This application claims the benefit of Great Britain Patent Application Number 2316544.2 filed on Oct. 30, 2023, the entire disclosure of which is incorporated herein by way of reference.

### FIELD OF THE INVENTION

The present techniques generally relate to a wearable sensor and manufacturing method for the wearable sensor. In particular, the present techniques provide a skin-conformable and compact wearable electronic apparatus for monitoring surface physiological and/or surface brain signals of the wearer and a manufacturing method for the apparatus. For example, the wearable electronic apparatus may be used to monitor electroencephalogram (EEG), electromyography (EMG) and/or electrocardiogram (ECG or EKG) signals.

### BACKGROUND OF THE INVENTION

Good quality ambulatory brain recordings are difficult to obtain because current techniques for monitoring human brain activity non-invasively are extremely susceptible to motion artefacts. Although devices exist which enable the collection of brain data outside clinical or laboratory environments, these devices require their wearer to refrain from making any head or body movements in order to acquire interpretable data. As such, it is currently difficult—without resorting to surgery—to monitor human brain activity effectively during most human behaviors. Solving this problem would, for instance, enable patients with neurological conditions to be monitored remotely, without interfering with their daily lives.

The present applicant has therefore identified the need for an improved sensor for monitoring physiological and/or brain activity data of users in real-world, ambulatory environments.

### SUMMARY OF THE INVENTION

The present techniques provide flexible, dry electrodes which may be used, for example, to provide skin-contacting electrodes of a wearable sensor. The dry electrodes may be fixedly and electrically coupled to electrodes of a wearable sensor, such that during use, the dry electrodes contact with skin of a human or animal wearer/user of the wearable sensor. The term “dry electrode” is used herein to mean electrodes which can be applied or adhered to skin without the use of any liquids or gels. For example, commonly used electrodes for EEG signal sensing may require a conductive electrolyte gel to be provided between the electrode and the skin, but gel-based electrodes cannot be self-applied and may not be suitable on skin having hair and/or for prolonged use. Dry electrodes do not require the use of such a gel, and are advantageously able to conform to skin.

The term “dry electrode” is used interchangeably herein with the terms “polymer electrode”, “electrode”, “conductive polymer electrode” and “electrically-conductive polymer electrode”.

The term “electrically-conductive polymer composition” is used interchangeably with the term “conductive polymer composition”.

Thus, in a first approach to the present techniques, there is provided a dry electrode for a wearable sensor, the dry electrode being formed from an electrically-conductive polymer composition, the electrically-conductive polymer composition comprising: an electrically-conductive material comprising particulate carbon; at least one additive; and a silicone polymer, wherein the particulate carbon is present in a range from 5 to 20 wt % by weight of the electrically-conductive polymer composition.

The conductive polymer composition is suitable for providing an interface between an electrode pad (which may be part of a sensor or wearable apparatus) and a user's skin. That is, the conductive polymer composition may reduce an electrode-skin impedance, such that an electrode/electrode pad that is coated with the conductive polymer composition is able to better measure electrophysiological signals from a user's skin.

Advantageously, a dry electrode formed using the conductive polymer composition may be dry, thin (e.g. <1 mm) and flexible, which enables the dry electrode to conform to a user's skin without requiring a liquid or gel electrolyte interface. Thus, the conductive polymer composition may be used to form a conductive polymer dry electrode that leaves little or no residue on a user's skin, can be reapplied several times and is comfortable for a user to wear.

Advantageously, a dry electrode formed using the conductive polymer composition may have a good balance between mechanical properties, such as mechanical conformability and/or flexibility, and electrical conductivity.

As noted above, the conductive polymer composition comprises an electrically-conductive material. The electrically-conductive material is used in the conductive polymer composition to impart conductivity to the composition. The electrically-conductive material comprises particulate carbon. Advantageously, the use of particulate carbon may result in a composition and/or dry electrode having good resistivity and electrical skin impedance values. For example, in some cases, the composition and/or dry electrode may advantageously have a resistivity value of about 2.2 Ω cm, an electrical skin impedance less than about 2000 kΩ, and simultaneously have excellent mechanical strength and wear properties.

The particulate carbon may be a conductive allotrope of carbon, such as, for example, graphene, carbon nanotubes (CNTs), and graphite such as natural graphite and artificial graphite. The particulate carbon may be doped or undoped. The particulate carbon may be any of: carbonaceous materials such as carbon black, acetylene black, Ketjenblack, channel black, furnace black, lamp black, thermal black and carbon fiber. These may be used alone or in a mixture of two or more.

On the proviso that particulate carbon is present (in the amounts defined herein), other electrically-conductive materials may also be used without particular limitation as long as they have electron conductivity without causing chemical change. Other electrically-conductive materials may be any of: metal powder, metal fibers or metal nanoparticles such as copper, nickel, aluminum and silver; conductive whiskers such as zinc oxide and potassium titanate; conductive metal oxides such as titanium oxide; electrically-conductive polymers; conductive powders; and polyphenylene derivatives. These may be used alone or in a mixture of two or more.

The particulate carbon material may be a high conductivity carbon material. An example high conductivity carbon material is carbon black or particulate carbon black. However, it will be understood that this is a non-limiting example of a high conductivity carbon material.

The conductive polymer composition comprises particulate carbon in a range from 5 wt. % to 20 wt. % by weight of the electrically-conductive polymer composition. The particulate carbon may be present in a range from 12 wt. % to 18 wt. %, preferably in a range from 13 wt. % to 17 wt. %, more preferably in a range from 14 wt. % to 17 wt. %, by weight of the conductive polymer composition.

The conductive polymer composition may comprise particulate carbon in a range from 9 wt. % to 16 wt. %, preferably in a range from 10wt. % to 15 wt. %, more preferably in a range from 12 wt. % to 15 wt. %, most preferably in a range from 14 wt. % to 15 wt. %, for example about 15 wt. % such as 14.9 wt. % or 15 wt. %, by weight of the conductive polymer composition.

The particulate carbon may be present in an amount of about 15 wt. % or 16 wt. % by weight of the conductive polymer composition.

The particulate carbon may be present in an amount of about 14.9 wt. % or 15 wt. % by weight of the conductive polymer composition. The particulate carbon may be present in an amount of about 16 wt. % or 16.1 wt. % by weight of the conductive polymer composition.

The present inventors have advantageously found that the use of particulate carbon in an amount ranging from 5 wt. % to 20 wt. % provides a good balance of electrical and mechanical properties. For instance, while adding higher levels of particulate carbon, i.e., above 20 wt. %, can increase conductivity, it has the disadvantage of reducing mechanical properties, including reduced flexibility and integrity of the electrode, leading to a poorer interface between the user's skin and the electrode and consequently increased skin impedance. Using less than 5 wt %, whist being lower cost, does not result in sufficiently even distribution of the particulate carbon throughout the composition.

The particulate carbon may be in any suitable form. The particulate carbon may be in the form of nanoparticles. By “nanoparticles” is meant particles having an average diameter in the range of nanometers (nm). Particulate carbon nanoparticles may have an average diameter up to 900 nm, such as up to 800 nm, such as up to 700 nm, such as up to 600 nm, such as up to 500 nm, such as up to 400 nm, such as up to 300 nm, such as up to 200 nm, such as up to 100 nm. Particulate carbon nanoparticles may have an average diameter ranging from 5 to 500 nm, such as from 10 to 300 nm, such as from 20 to 250 nm, such as from 30 to 200 nm, such as from 40 to 150 nm, such as from 50 nm to 100 nm. Advantageously, the use of nanoparticles may allow a substantially even distribution of the particulate carbon throughout the composition, even at low concentrations. Advantageously, the use of nanoparticles may positively impact the electrical and mechanical performance of the dry electrode. For example, the use of nanoparticles, which have a higher surface area compared to larger particles (i.e., on the micro-scale or larger), may decrease skin impedance, increase electrical conductivity, increase mechanical robustness and/or increase wear resistance. These advantages can be achieved at relatively low cost.

As noted above, the conductive polymer composition comprises at least one additive. The at least one additive may be, for example, a surfactant. The electrically-conductive polymer composition may comprise a surfactant to improve the miscibility of the composition and/or to improve mechanical properties of the composition such as softness. The surfactant may be any suitable surfactant. For example, the surfactant may be an ionic or non-ionic surfactant. Preferably, the surfactant may be a non-ionic surfactant. The surfactant may be selected from any one of: those sold under the tradename Triton (commercially available from Dow), such as Triton X-100; those sold under the tradename Tween (commercially available from Croda), such as Tween 80, Tween 20, Tween 40, Tween 60 and/or mixtures thereof. These may be used alone or in a mixture of two or more. The surfactant may comprise Triton X-100 (also known as 2-[4-(2,4,4-trimethylpentan-2-yl)phenoxy]ethanol). The surfactant may comprise Tween 80 (also known as polyoxyethylene (20) sorbitan monooleate). However, it will be understood that these are a non-limiting example surfactant and that other surfactants may be used which have substantially similar properties.

In some cases, the at least one additive may be Triton X-100, and the Triton X-100 may be present in a range from 1wt. % to 30 wt. %, preferably in a range from 1wt. % to 20 wt. %, more preferably in a range from 5 wt. % to 15 wt. %, for example about 10wt. % such as 10wt. %, by weight of the conductive polymer composition.

In some cases, the at least one additive may be Tween 80, and the Tween 80 may be in a range from 1wt. % to 30 wt. %, preferably in a range from 1wt. % to 20 wt. %, more preferably in a range from 5 wt. % to 15 wt. %, for example about 10wt. % such as 10 wt. %, by weight of the conductive polymer composition. Advantageously, Tween has the advantage of reduced electrode toxicity.

As noted above, the conductive polymer composition comprises a silicone polymer. The silicone polymer may comprise an elastomer base and a curing agent. That is, the silicone polymer may be provided in two parts, and a curing process of the silicone polymer may only start when a curing agent is added to the silicone polymer base. It will be understood by a person skilled in the art that the silicone polymer, or polymer mixture, prior to the curing process will be uncured, and that after the curing process the silicone polymer may be crosslinked, suitably via a curing agent, to form a crosslinked polymer network in the final adhesive polymer composition. For the avoidance of doubt, by “uncured” is meant that the silicone polymer has not yet reacted with a curing agent (or otherwise) to form a crosslinked polymer network. The term “uncured adhesive composition” is herein used interchangeably with “adhesive polymer mixture” and “uncured adhesive polymer mixture”.

Thus, the present techniques extend to a polymer mixture for forming an electrically-conductive polymer composition, the polymer mixture comprising an electrically-conductive material comprising particulate carbon; at least one additive; a silicone polymer; and a curing agent, wherein the particulate carbon is present in a range from 5 wt. % to 20 wt. % by weight of the polymer mixture.

Advantageously, providing the silicone polymer in two parts allows other components to be mixed with the elastomer base before a curing process starts, ensuring even distribution of any other components. The silicone polymer may preferably be inert and non-toxic, to ensure the conductive polymer composition is suitable for contact with human or animal skin. The silicone polymer may be any suitable material. The silicone polymer may be polydimethylsiloxane (PDMS). PDMS is an example of an elastomer, and it will be understood that any other suitable elastomer may be used, as long as they are also inert and non-toxic. For example, other non-limiting silicone-based elastomers include natural rubbers, and silicone

On the proviso that a silicone polymer is present, other polymer materials may also be used without particular limitation as long as they have electron conductivity without causing chemical change. For example, non-limiting examples include polyurethanes.

As noted above, the conductive polymer composition comprises at least one additive. Thus, in some cases, the conductive polymer composition may comprise, at least, a first additive and a second additive. The first additive may be a surfactant, as noted above. The second additive may be a material which provides the conductive polymer composition with improved electrical conductivity properties. For example, the second additive may be ethylene glycol.

In cases where the second additive is ethylene glycol, the ethylene glycol may be in a range from 1wt. % to 30 wt. %, preferably in a range from 1wt. % to 20 wt. %, more preferably in a range from 3 wt. % to 15 wt. %, most preferably in a range from 6 wt. % to 8 wt. %, for example about 7 wt. % such as 7 wt. %, by weight of the conductive polymer composition.

As noted above, the electrically-conductive polymer composition may be used to improve the connectivity and conductivity between a skin-contacting component of a wearable sensor, and skin of a human or animal. Preferably, an electrical skin impedance of the conductive polymer composition (at 30 Hz) may be in a range from 100 kD to 2000 kΩ, preferably in a range from 100 kΩ to 1000 kΩ, more preferably in a range from 50 kΩ to 500 kΩ, most preferably in a range from 10 kΩ to 200 kΩ.

In a second approach to the present techniques, there is provided an uncured electrically-conductive composition (or polymer mixture) for a dry electrode, the uncured electrically-conductive composition (or polymer mixture) comprising: an electrically-conductive material comprising particulate carbon; at least one additive; a silicone polymer; and a curing agent, wherein the particulate carbon is present in a range from 5 wt. % to 20 wt. % by weight of the electrically-conductive polymer composition.

The features described above with respect to the first approach apply equally to the second approach and therefore, for the sake of conciseness, are not repeated.

The curing agent may be any suitable curing agent, and may depend on the silicone polymer.

In a particular example, the electrically-conductive material may be particulate carbon, the at least one additive may be Triton X-100 and/or Tween 80, the silicone polymer may be uncured polydimethylsiloxane, and the curing agent may be the curing agent for polydimethylsiloxane. By “the” curing agent for polydimethylsiloxane is meant any suitable curing agent (as per above).

In a third approach to the present techniques, there is provided a method for manufacturing an electrically-conductive polymer electrode, the method comprising: obtaining an electrically-conductive polymer mixture by mixing an electrically-conductive material comprising particulate carbon, at least one additive, a silicone polymer, and optionally a curing agent; and curing the conductive polymer mixture to obtain a cured electrically-conductive polymer, wherein the particulate carbon is present in a range from 5 wt. % to 20 wt. % by weight of the polymer mixture. The cured electrically-conductive polymer composition may be used to form a dry electrode, for use in, for example, a wearable sensor.

Features described above with respect to the first approach apply equally to the third approach and therefore, for the sake of conciseness, are not repeated.

In a particular example, obtaining the conductive polymer mixture may comprise: mixing particulate carbon, at least one additive, such as a surfactant, and a silicone polymer; and adding a curing agent to obtain the conductive polymer mixture. That is, the silicone polymer may be provided in two parts, and a curing process of the silicone polymer may only start when a curing agent is added to the silicone polymer. Advantageously, this allows other components to be mixed with the elastomer before a curing process starts, ensuring even distribution of any other components.

Curing the conductive polymer mixture may comprise at least one thermal curation step at a predetermined curation temperature. Controlling a temperature of a thermal curation step ensures that the curing process is controlled and therefore, along with controlling a ratio of a curing agent in the conductive polymer, allows control over the ratio of cured to uncured polymer chains in the cured conductive polymer composition. In turn, control over the ratio of cured to uncured polymer chains means that the properties of the conductive polymer can be controlled. For example, a larger amount of cured chains results in a more solid conductive polymer, whereas less cured chains result in a softer conductive polymer. These are important properties, as the softness of the conductive polymer and other surface properties of the conductive polymer determine how well the conductive polymer conforms to a user's skin and thus how well the conductive polymer works as an interface between the electrode pad and the user's skin.

Manufacturing a conductive polymer electrode may further comprise patterning a surface of the conductive polymer to obtain an increased surface area of the conductive polymer. An increased surface area of the patterned conductive polymer in turn ensures that a contact surface between the conductive polymer electrode and a user's skin is increased, which in turn causes electrode-skin impedance to be decreased. An increased contact surface improves the quality of an electrophysiological signal that can be measured by the at least one electrode pad via the conductive polymer electrode interface.

In a fourth approach to the present techniques, there is provided a wearable sensor for monitoring physiological and/or brain signals, the sensor comprising: at least one electrode pad for monitoring physiological and/or brain signals, wherein the at least one electrode pad is at least partially coated in a coating formed of the conductive polymer composition as described herein.

The wearable sensor may comprise circuitry coupled to the at least one electrode pad. Preferably, the wearable sensor may comprise at least two electrode pads, where one electrode pad is used as a reference and another electrode pad is used to sense a signal. The circuitry may all be contained in a flexible (or rigid) PCB (see the readout electronics module described below with reference to the Figures). The at least one electrode pad may interface with the circuitry/readout electronics module via a connector. In some cases, the at least one electrode pad may be directly coupled to the circuitry.

The coating may comprise patterning to obtain an increased surface area of the conductive polymer. This may improve the ability for the coating (i.e. dry electrode) to contact the skin and sense signals.

The at least one electrode pad (and the coating) and the circuitry (where present) may be provided on a thin flexible substrate, and the circuitry (where present) may also be flexible. The substrate may have a thickness in a range from 0.04 to 0.3 mm. For example, the substrate may have a thickness of 0.08 mm. The substrate may be made from flexible materials, and may comprise, for example, a polymer and/or polyimide layer and/or conductive tracks (e.g. formed from copper) with a thickness in a range from 10 to m, for example. For example, the conductive tracks may be formed of copper and have a thickness of 18 μm. At least part of the substrate may have an immersion gold surface finish. A thin flexible substrate (and flexible circuitry, where present) mean that the sensor adapts to a user's movement—the sensor moves along with the user's skin, even when the user is moving. Advantageously, this means that contact between a conductive polymer electrode interface and the user's skin is always maintained. Additionally, in cases where the circuitry is flexible and provided on the flexible substrate, the circuitry will also move along with any movements of the user. Further advantageously, the ability for the sensor and circuitry to move with the electrode pads eliminates any motion artefacts that may appear on the measured signal as a result of any movement of the circuitry relative to the electrode pads. Thus, a thin conductive polymer electrode, a flexible substrate and flexible circuitry improve a signal quality of an electrophysiological signal measured by the electrode pads.

In a fifth approach of the present techniques, there is provided a method for manufacturing at least one electrode of a wearable sensor for monitoring physiological and/or brain signals, the wearable sensor having at least one electrode pad, wherein the method comprises: coating at least part of at least one electrode pad in an electrically-conductive polymer mixture, the mixture comprising an electrically-conductive material comprising particulate carbon, at least one additive, and a silicone polymer, wherein the particulate carbon is present in a range from 5 wt. % to 20 wt. % by weight of the electrically-conductive material; and curing the electrically-conductive polymer mixture to obtain a cured electrically-conductive polymer on the at least one electrode pad. In this way, a dry electrode is formed on each electrode pad, and together, each dry electrode and electrode pad pair form an electrode of the wearable sensor.

In a particular example, the method may comprise adding a curing agent to the polymer mixture. Adding a curing agent to the silicone polymer may start the curing process (as described herein). That is, the silicone polymer may be provided in two parts, and the curing process of the silicone polymer may only start when a curing agent is added to the silicone polymer.

The method may further comprise patterning a surface of the conductive polymer mixture, prior to curing, to obtain an increased surface area of the cured conductive polymer (i.e. of the cured coating).

### BRIEF DESCRIPTION OF THE DRAWINGS

Implementations of the present techniques will now be described, by way of example only, with reference to the accompanying drawings, in which:

FIG. 1A is a plan view of an example layout of a sensor for measuring surface physiological and/or surface brain signals;

FIG. 1B shows the sensor of FIG. 1A which has flexible dry electrodes;

FIG. 2A shows a top and side perspective view of an example pattern that may be applied to the dry electrodes of FIG. 1B;

FIG. 2B shows a side view of an example pattern that may be applied to the dry electrodes of FIG. 1B;

FIG. 3 is a flowchart showing the steps involved in producing a conductive polymer composition for use as a dry electrode;

FIG. 4 is a diagram showing an overview of the process for coating the F-PCB of the sensor with the conductive polymer electrode composition; and

FIG. 5 is a block diagram of a system comprising the sensor and a readout electronics device.

FIG. 6 is a line graph showing the relationship between the concentration of particulate carbon (carbon black) by weight percent and the resistivity (blue, or dark shade, line); and the relationship between the concentration of carbon black by weight percent and skin impedance (yellow, or light shade, line).

### DETAILED DESCRIPTION OF THE PREFERRED EMBODIMENTS

Broadly speaking, embodiments of the present techniques provide a skin-conformable electrode array and compact wearable electronic apparatus for monitoring surface physiological and/or surface brain signals of the wearer. In particular, the present techniques provide flexible, dry electrodes which may be used, for example, to provide skin-contacting electrodes of a wearable sensor. The dry electrodes may be fixedly and electrically coupled to electrodes of a wearable sensor, such that during use, the dry electrodes contact with skin of a human or animal wearer/user of the wearable sensor.

Two causes of motion artefacts in electroencephalography are displacement of sensors relative to the skin and/or interruption or alteration of the quality of electrical contact between the skin and the sensor, and cable sway introducing electrical noise into analogue signals.

The present techniques solve the problem of motion artefacts by providing dry electrode-based sensors on a flexible printed circuit board, instead of using rigid materials and/or liquid gel electrodes to build the sensors. These flexible dry electrodes are able to conform to the skin's surface, establishing perfect contact with the skin. These sensors have low mass, meaning by using an adhesive or another method of bonding them to the skin, they can remain in place even during vigorous movement.

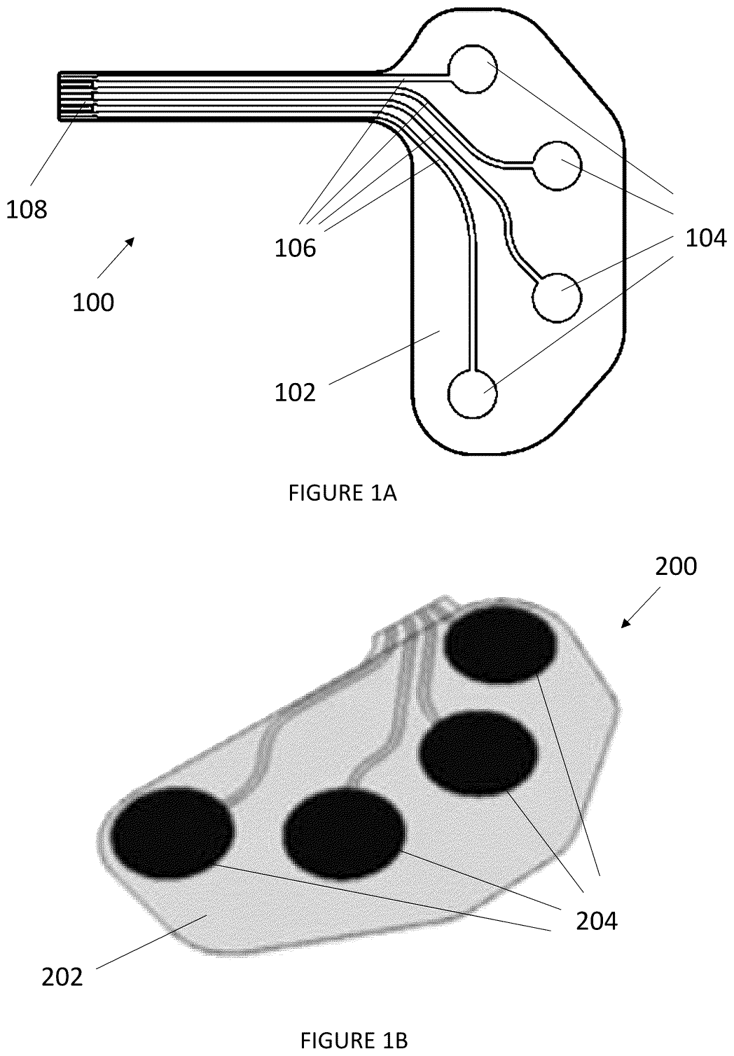

FIG. 1A is a plan view of an example layout of a sensor for measuring surface physiological and/or surface brain signals. The sensor 100 may comprise a substrate, such as, for example, a flexible printed circuit board (F-PCB) 102 on which at least one electrode pad 104 and at least one conductive track 106 are deposited. Preferably, the wearable sensor may comprise at least two electrode pads, where one electrode pad is used as a reference and another electrode pad is used to sense a signal. The sensor may be used for electrophysiological measurements such as EEG, ECG and/or EMG (which are the measurements most susceptible to motion-induced noise) or measurement of any other surface electrical signal from the body including, but not limited to, electro-oculogram (EOG), electro-gastrogram (EGG), electro-spinogram (ESG) or electro-olfactogram (EOFG). When the sensor is provided on a user's head, the at least one electrode pad may sense surface brain signals. In this case, the at least one electrode pad may be able to provide (directly or indirectly) information on the cognition, emotional state, or disease indicators. The at least one conductive track 106 may transmit measurements from the at least one electrode pad to a connector 108. In the example shown in FIG. 1A, the sensor 100 of the present techniques is advantageously not affected by cable sway. This is because circuits and connections between electrical elements of the apparatus are made through conductive tracks 106 deposited onto the flexible circuit board 102 itself, which moves with the skin. In contrast, existing devices use cables, which move relative to the skin and thus introduce motion artefacts into the signal. The sensor 100 may also comprise a connector 108 on the F-PCB 102. The connector 108 may be used to connect the sensor 100 to an apparatus and/or readout electronics for receiving a physiological signal that is measured by the sensor 100.

The substrate may be thin and flexible. The substrate may have a thickness in a range from 0.04 to 0.3 mm. For example, the substrate may have a thickness of 0.08 mm. The substrate may be made from flexible materials, and may comprise a polymer and/or polyimide and/or metal conductive tracks (e.g. copper) with a thickness in a range from 10 to 25 μm, for example, copper with a thickness of 18 μm. At least part of the substrate may have an immersion gold surface finish. When the substrate is a flexible printed circuit (F-PCB), the substrate may comprise the following layers: a polyimide substrate, copper, an electroless nickel immersion gold (ENIG) finish, and a polyimide coverlay (i.e. solder mask).

The copper layer may have a finished thickness of 18 ym, the thin electroless nickel immersion gold (ENIG) finish may have a thickness of 3 rack units (U″) and the polyimide coverlay (i.e. solder mask) may have a thickness in a range from 5 to 20 μm, preferably in a range from 10 to 15 μm, for example, about 13 μm such as 12.5 μm or 13 μm. The overall F-PCB substrate stack may have a thickness in a range from 0.04 to 0.3 mm, for example, a thickness of 0.08 mm

The at least one electrode pad 104 may be a gold electrode pad, which is highly conductive and highly resistant to corrosion. Additionally or alternatively, the at least one electrode pad 104 may be made from any other suitable highly conductive material, such as stainless steel, iridium, titanium, silver or another suitable metal, alloy or other material. The at least one electrode pad 104 may be placed at any suitable position on the F-PCB 102. When there is more than one electrode pad 104, the electrode pads 104 may be arranged in any suitable configuration. For example, the location of the electrode pads 104 may be adapted such that, in use, the electrode pads are placed at physiologically sensible locations on a user's body. For example, the electrode pads may be arranged such that the sensor may be ideally placed to measure a user's brain activity. Additionally or alternatively, the shape, size, number and placement of the electrode pads may be varied such that the sensor fits a variety of user groups or electrophysical monitoring functions. For example, the sensor, and accordingly the electrode pads may be smaller or larger to fit smaller (such as children) or large head sizes, or to have a different shape and size and/or number of electrode pads in order to serve as an electrode array for EKG, EEG or EMG, for example. The PCB 102 may take any suitable shape. The shape and design of the sensor, and therefore the placement of the electrode pads 104, may depend on where the sensor is to be used and the shape and size of this location. Maximizing user experience, i.e. reducing pain and discomfort, may be taken into consideration in the shape and design of the sensor.

FIG. 1B shows the sensor of FIG. 1A which has flexible dry electrodes. The dry electrodes are also referred to herein as polymer electrodes, conductive polymer electrodes or electrically-conductive polymer electrodes. The sensor 200 comprises at least one dry electrode 204 which may be provided on at least one electrode pad (not visible here as the electrode pads are below the dry electrodes 204, but see FIG. 1A) of the flexible printed circuit board. While the electrode pad of the F-PCB may be highly conductive, in order for the electrode pads to be used for surface electrophysiology (EEG, EMG or ECG for example) on the skin, a conductive interface between the F-PCB electrode pads and the skin is necessary. This is because otherwise skin-electrode impedance is too high. In other words, it is very difficult/impossible to ensure that the electrode of the F-PCB makes full contact with the skin at all times without using a gel electrolyte interface. It is especially difficult to ensure sufficient contact when a user wearing the sensor moves. However, gel electrolyte interfaces are not desired, for the reasons explained above.

Thus, the present techniques provide an electrically-conductive polymer composition for a dry electrode 204, the conductive polymer composition comprising: an electrically-conductive material comprising particulate carbon; at least one additive; and a silicone polymer, wherein the particulate carbon is present in a range from 5 wt. % to 20 wt. % by weight of the conductive polymer composition.

The conductive polymer composition is suitable for providing a dry electrode, i.e. an interface between an electrode pad 104 (which may be part of a sensor or wearable apparatus 100) and a user's skin. That is, the conductive polymer composition may reduce an electrode-skin impedance, such that an electrode/electrode pad 104 that is coated with the conductive polymer composition is able to better measure electrophysiological signals from a user's skin. Advantageously, a dry electrode 204 formed using the conductive polymer composition may be dry, thin (e.g. <1 mm in thickness) and flexible, meaning that the dry electrode 204 conforms to a user's skin without requiring a liquid or gel electrolyte interface. Thus, the conductive polymer composition may form a dry electrode 204 that leaves little or no residue on a user's skin, can be reapplied and cleaned several times and is comfortable for a user to wear.

As noted above, the conductive polymer composition comprises an electrically-conductive material comprising particulate carbon. The electrically-conductive material is used in the conductive polymer composition to impart conductivity to the composition. The particulate carbon may be a conductive allotrope of carbon, such as, for example, graphene, carbon nanotubes (CNTs), and graphite such as natural graphite and artificial graphite. The particulate carbon may be doped or undoped. The particulate carbon may be any of: carbonaceous materials such as carbon black, acetylene black, Ketjenblack, channel black, furnace black, lamp black, thermal black and carbon fiber. These may be used alone or in a mixture of two or more. On the proviso that particulate carbon is present (in the amounts defined herein), other electrically-conductive materials may also be used without particular limitation as long as they have electron conductivity without causing chemical change. Other electrically-conductive materials may be any of: metal powder, metal fibers or metal nanoparticles such as copper, nickel, aluminum and silver; conductive whiskers such as zinc oxide and potassium titanate; conductive metal oxides such as titanium oxide; electrically-conductive polymers; conductive powders; and polyphenylene derivatives. These may be used alone or in a mixture of two or more.

As noted above, the particulate carbon material may be a high conductivity carbon material. An example high conductivity carbon material is carbon black or particulate carbon black. However, it will be understood that this is a non-limiting example of a high conductivity carbon material.

As noted above, the conductive polymer composition comprises particulate carbon in a range from 5 wt. % to 20 wt. % by weight of the electrically-conductive polymer composition. The particulate carbon may be present in a range from 12 wt. % to 18 wt. %, preferably in a range from 13 wt. % to 17 wt. %, more preferably in a range from 14 wt. % to 17 wt. %, by weight of the conductive polymer composition. The particulate carbon may be present in an amount of about 15 wt. % or 16 wt. % by weight of the conductive polymer composition. The particulate carbon may be present in an amount of about 14.9 wt. % or 15 wt. % by weight of the conductive polymer composition. The particulate carbon may be present in an amount of about 16 wt. % or 16.1 wt. % by weight of the conductive polymer composition.

As noted above, the particulate carbon may be in the form of nanoparticles. By “nanoparticles” is meant particles having an average diameter in the range of nanometers (nm). Particulate carbon nanoparticles may have an average diameter up to 900 nm, such as up to 800 nm, such as up to 700 nm, such as up to 600 nm, such as up to 500 nm, such as up to 400 nm, such as up to 300 nm, such as up to 200 nm, such as up to 100 nm. Particulate carbon nanoparticles may have an average diameter ranging from 5 to 500 nm, such as from 10 to 300 nm, such as from 20 to 250 nm, such as from 30 to 200 nm, such as from 40 to 150 nm, such as from 50 nm to 100 nm.

As noted above, the conductive polymer composition comprises at least one additive. The at least one additive may be, for example, a surfactant. The electrically-conductive polymer composition may comprise a surfactant to improve the miscibility of the composition, to improve electrical conductivity of the composition, and/or to improve mechanical properties of the composition. The surfactant may be any suitable surfactant. For example, the surfactant may be an ionic or non-ionic surfactant. Preferably, the surfactant may be a non-ionic surfactant. The surfactant may be selected from any one of: those sold under the tradename Triton (commercially available from Dow), such as Triton X-100; those sold under the tradename Tween (commercially available from Croda), such as Tween 80, Tween 20, Tween 40, Tween 60 and/or mixtures thereof. These may be used alone or in a mixture of two or more. The surfactant may comprise Triton X-100 (also known as 2-[4-(2,4,4-trimethylpentan-2-yl)phenoxy]ethanol). The surfactant may comprise Tween 80 (also known as polyoxyethylene (20) sorbitan monooleate). However, it will be understood that these are a non-limiting example surfactant and that other surfactants may be used which have substantially similar properties.

In some cases, the at least one additive may be Triton X-100, and the Triton X-100 may be in a range from 1wt. % to 30 wt. %, preferably in a range from 1wt. % to 20 wt. %, more preferably in a range from 5 wt. % to 15 wt. %, for example about 10wt. % such as 10wt. %, by weight of the conductive polymer composition.

In some cases, the at least one additive may be Tween 80, and the Tween 80 may be in a range from 1wt. % to 30 wt. %, preferably in a range from 1wt. % to 20 wt. %, more preferably in a range from 5 wt. % to 15 wt. %, for example about 10wt. % such as 10 wt. %, by weight of the conductive polymer composition.

As noted above, the conductive polymer composition comprises a silicone polymer. The silicone polymer may comprise an elastomer base and a curing agent. That is, the silicone polymer may be provided in two parts, and a curing process of the silicone polymer may only start when a curing agent is added to the silicone polymer base. Advantageously, this allows other components to be mixed with the elastomer base before a curing process starts, ensuring even distribution of any other components. The silicone polymer may preferably be inert and non-toxic, to ensure the conductive polymer composition is suitable for contact with human or animal skin. The silicone polymer may be polydimethylsiloxane (PDMS). PDMS is an example of an elastomer, and it will be understood that any other suitable elastomer may be used, as long as they are also inert and non-toxic.

The silicone polymer may comprise an elastomer base and a curing agent. That is, the silicone polymer may be provided in two parts, and a curing process of the silicone polymer may only start when a curing agent is added to the silicone polymer. Advantageously, this allows other components to be mixed before a curing process starts, ensuring even distribution of any other components.

As noted above, the conductive polymer composition comprises at least one additive. Thus, in some cases, the conductive polymer composition may comprise, at least, a first additive and a second additive. The first additive may be a surfactant, as noted above. The second additive may be a material which provides the conductive polymer composition with improved electrical conductivity properties. For example, the second additive may be ethylene glycol.

In cases where the second additive is ethylene glycol, the ethylene glycol may be in a range from 1wt. % to 30 wt. %, preferably in a range from 1wt. % to 20 wt. %, more preferably in a range from 3 wt. % to 15 wt. %, most preferably in a range from 6 wt. % to 8 wt. %, for example about 7 wt. % such as 7 wt. %, by weight of the conductive polymer composition.

As noted above, the electrically-conductive polymer composition may be used to improve the connectivity and conductivity between a skin-contacting component of a wearable sensor, and skin of a human or animal. Preferably, an electrical skin impedance of the conductive polymer composition (at 30 Hz) may be in a range from 100 kD to 2000 kΩ, preferably in a range from 100 kΩ to 1000 kΩ, more preferably in a range from 50 kΩ to 500 kΩ, most preferably in a range from 10 kΩ to 200 kΩ.

As shown in FIG. 1B, thin (e.g. <1 mm) films of the electrically-conductive polymer composition may be provided on top of the F-PCB electrode pads 104. The conductive polymer composition may cover part of or all of the electrode pad(s) 104 of the sensor 100.

Conductive polymers have favorable electrical (high conductivity) and physical (stretchability, flexibility, give) properties. Thus, using a conductive polymer composition to form a dry electrode allows the dry electrode to follow the surface of the skin very closely due to the flexibility, stretchability and “sponginess” of the dry electrode. This enables the dry electrode to achieve relatively low (<80-1000 kΩ) electrode-skin impedances without using electrolyte gel. Compared to any other type of dry electrode, the favorable physical properties of the conductive polymer electrode mean that there is far less variability in electrode-skin impedance from sensor to sensor. This has a great impact on an achievable signal to noise ratio (SNR) when measuring physiological signals using the dry electrodes. This is because minimizing variability means that the actual common mode rejection ratio achievable by a differential amplifier is maximized. The differential amplifier may be applied when receiving a signal measured by the sensor.

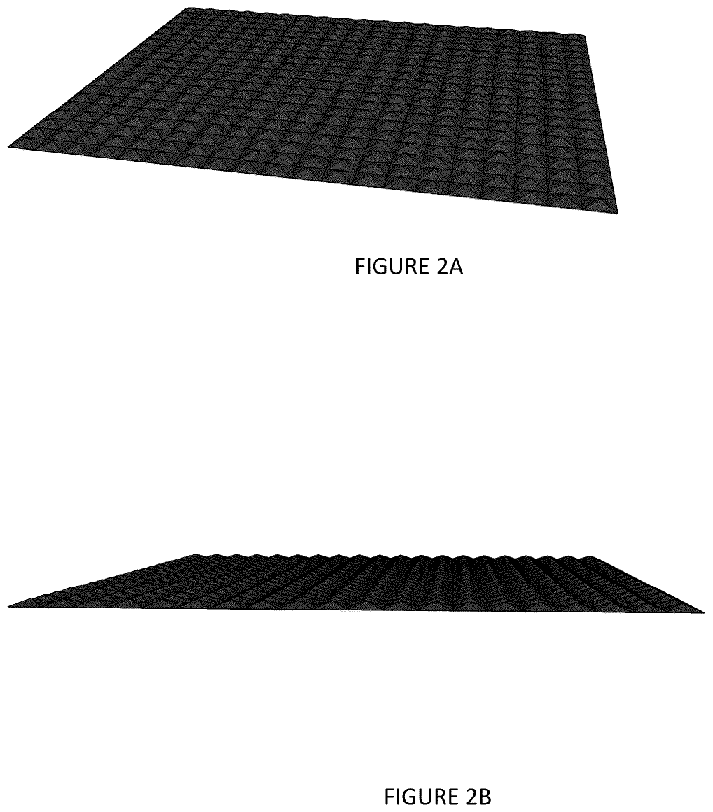

FIGS. 2A and 2B show an example pattern that may be applied to the dry electrodes of FIG. 1B. Thus, a surface of the dry electrode(s) may comprise patterning to obtain an increased surface area of the dry electrode. An increased surface area of the patterned dry electrode in turn ensures that a contact surface between the dry electrode and a user's skin is increased. Specifically, the effective area of contact between the electrode and the skin may be increased, which further lowers electrode-skin impedance. An increased contact surface improves the quality of an electrophysiological signal that can be measured by the at least one electrode pad 104 via the dry electrode 204 interface. Thus, an increase in surface area of the conductive polymer electrode improves a signal quality of the measured electrophysiological signal.

The pattern may be a 3D pattern of pyramids, as shown in FIGS. 2A and 2B, with 40 μm height and 200 μm centre-to-centre spacing. It will be understood that the pattern shown in FIGS. 2A and 2B is merely exemplary and non-limiting, and that any other suitable pattern may be used. For example, the pattern may be a 3D pattern of cubes, prisms or hemispheres. The individual elements of the pattern may vary in size and the sizes above are merely an example of suitable sizes. Other suitable sizes may be on the order of μm. In particular, the size of the individual elements of the pattern may be on the order of magnitude of the roughness of human skin, which is on the order of magnitude of dozens to hundreds of microns.

The pattern may, for example, be applied using a stamp. The stamp may, for example, be made from a polymer, such as polyurethane. For example, the stamp may be fabricated using a Polyuretane Ecoflex 00-30 kit by dispensing the same amount of PartA and Part B, 10 g of each, into a container (1A:1B by weight). Part A and Part B may then be mixed thoroughly for three minutes. Finally, the mixture may be poured it to a suitable box, for example, a plastic box. The mixture may be poured or spread out such that, after curing, the mixture forms an approximately 5 mm thick membrane. Curing may happen overnight at room temperature. After, the, for example, polyurethane membrane is cured, the membrane may be removed from the box. The pattern may be formed in the membrane using a laser, such as a CO₂ laser. As mentioned above, the pattern may be designed of a 2D pattern of squares with a 200×200 m base area, resulting in a 3D pattern of pyramids with 60 μm height with 200 μm centre-to-centre spacing. The pattern may be made in Adobe Illustrator, for example, and applied using a Laser System VLS3.50: Cutting and Engraving Machine with the following parameters: power of 5%, speed of 100%, PPI of 1000. After forming the pattern using the laser, the membrane should be cleaned with Isopropyl alcohol (IPA) and Deionised (DI) water. Then, the stamp formed of the membrane may be placed on the uncured conductive polymer electrodes to apply the pattern to the conductive polymer electrodes. The pattern may be applied by applying pressure to the stamp by hand, or by any other suitable method. The stamp is then carefully removed from the conductive polymer electrodes. The resulting height of the pyramids is reduced in the conductive polymer electrode compared to the height of the pyramids in the stamp. For example, the conductive polymer electrode pattern may comprise pyramids of m height. The height of the pyramids may be in the range of skin roughness, which thereby helps the conductive polymer electrodes to conform to the skin.

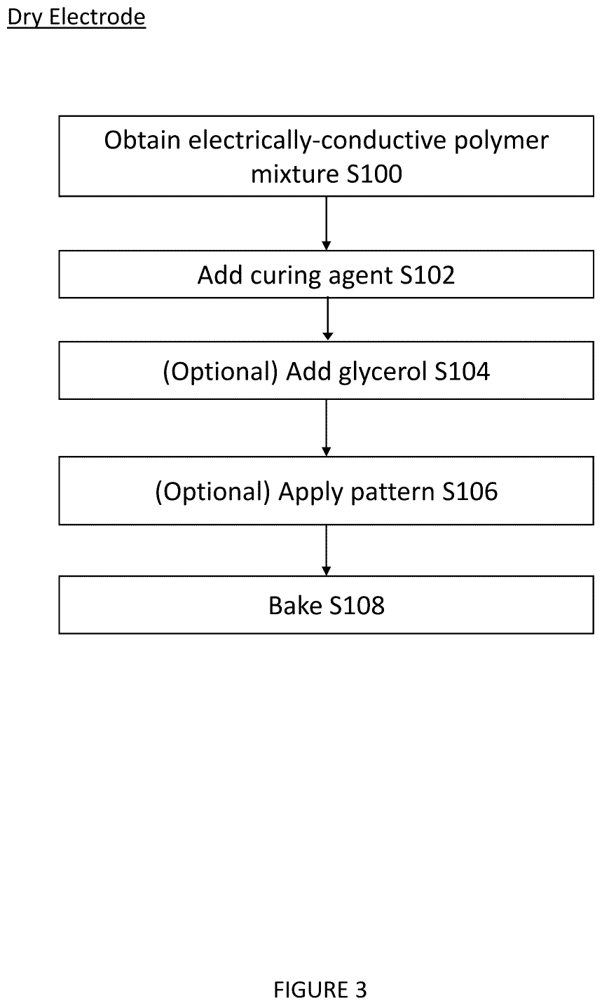

FIG. 3 is a flowchart showing the steps involved in producing the conductive polymer composition that may be used to form dry electrodes. The method comprises: obtaining an electrically-conductive polymer mixture (step S100). Obtaining, at step S100, the electrically-conductive polymer mixture may comprise: mixing an electrically-conductive material (e.g. one of the electrically-conductive materials described above), at least one additive (such as a surfactant or any of the additives described above), and a silicone polymer (or any other suitable silicone oil-based material described above).

The method comprises adding a curing agent (step S102) to cure the electrically-conductive polymer mixture. That is, the silicone polymer may be provided in two parts, and a curing process of the silicone polymer may only start when a curing agent is added to the silicone polymer. Advantageously, this allows other components to be mixed with the elastomer before a curing process starts, ensuring even distribution of any other components.

The method then comprises thermally curing, such as by baking or otherwise heating, the polymer mixture (step S108). The cured electrically-conductive polymer may form a dry electrode on top of an electrode pad 104, for use in, for example, a wearable sensor 100.

### Example 1

A specific example of a dry electrode is now described, for illustrative purposes.

In this specific example, step S100 comprises mixing carbon, the silicone polymer base and at least one additive. For example, the following amounts of each substance may be added: 0.74 g (15 wt %) of carbon black, 3.3 g of PDMS part A, 0.35 g of ethylene glycol and 0.5 g of Triton X-100 and gently hand mixed for approximately five minutes, until all the carbon black is involved. Using 15w % of carbon black may achieve best conductivity of the conductive polymer. Using between 12-15 w % carbon black achieved good conductivity of the conductive polymer. That is 0.74 g of carbon black may be added to the above mixture. Alternatively, when adding 12w % of carbon black, 0.59 g (12 wt %) of carbon black may be added to the same amounts of other components.

Triton X-100 is a non-ionic surfactant that improves miscibility of carbon black and PDMS and thus improves the mechanical properties of the conductive polymer. That is, Triton X-100 acts as a ‘softener’, and depending on the amount of Triton X-100, this results in spongier or more solid electrodes.

Ethylene Glycol is a polar organic compound that works as a secondary dopant and thus improves the electrical properties of the conductive polymer.

Next, a curing agent may be added to the mixture at step S102. Using the quantities specified above, after obtaining a uniform ‘paste’, add 0.07 g of PDMS part B to the mixture and mix again for five minutes. PDMS part B is the curing agent for PDMS and is added only at the end to avoid the mixture curing during the fabrication process, ensuring a better uniformity of the final paste.

The silicone polymer may be polydimethylsiloxane which is created when PDMS Part A (silicone polymer base) and Part B (curing agent) react and cure. When heated, PDMS Part A and Part B crosslink, forming a network structure of chemical bonds.

Optionally, as shown in FIG. 3, glycerol may be added at this stage (step S104). Adding glycerol as a further additive may improve the adhesive properties of the conductive polymer electrode. For example, 0.55 g of glycerol may be added to the mixture with quantities specified above at this stage if using 14-15 wt % of carbon black or 1 g (17 wt %) of glycerol if using 12-14 wt % of carbon black. Alternatively, when adding glycerol, 0.66 g may be added to the composition, with the above specified quantities of the other components.

Thus, to summarize, the following components may be used to obtain the example electrically-conductive polymer electrode composition described above:
Carbon black (C), C-Nergy Super C65, ≥99.975, PI-KEM
Ethylene Glycol—RPE (CH₂OHCH₂OH), 99.5% (GLC), Carlo Erba Reagents
Triton X-100 ((C₂H₄O)ₙC₁₄H₂₂O), alkylaryl polyether alcohol, ≤100%, BAKER ANALYZED, J.T.Baker®
Polydimethylsiloxane ((C₂H₆OSi)n), Sylgard 184 Silicone Elastomer Kit, Dow:
Part A—Sylgard 184 Silicone Elastomer Base:
Dimethyl siloxane, dimethylivinyl terminated—68083-19-2
Dimethylvinylated and trimethylated silica—68988-89-6
Tetra (trimethoxysiloxy) silane—3555-47-3
Ethyl benzene—100-41-4
Part B—Sylgard 184 Silicone Elastomer Curing Agent:
Dimethyl, methylhydrogen siloxane—68037-59-2
Dimethyl siloxane, dimethylvinyl terminated—68083-19-2
Dimethylvinylated and trimethylated silica—68988-89-6
Tetramethyl tetravinyl cyclotetra siloxane—2554-06-5
Ethyl benzene—100-41-4
Glycerol (HOCH₂CH(OH)CH₂OH), BioXtra, >=99% (GC), Sigma Aldrich

The components of the conductive polymer may be present in the conductive polymer in the following proportions:

| | | w/w | |
|---|---|---|---|
| Component | Mass (g) | (solute/solution) % | Range± (g) |
| PDMS part A (base) | 3.3 | 66.532 | ±0.3 |
| PDMS part B | 0.07 | 1.411 | ±0.04 |
| (curing agent) | | | |
| Carbon black | 0.74 | 14.919 | ±0.07 |
| Ethylene Glycol | 0.35 | 7.056 | ±0.09 |
| Triton X-100 | 0.5 | 10.081 | ±0.04 |

### Example 2

A specific example of a dry electrode is now described, for illustrative purposes.

In this specific example, step S100 comprises mixing carbon, the silicone polymer base and at least one additive. For example, the following amounts of each substance may be added: 0.80 g (16 wt %) of carbon black, 3.59 g of PDMS part A, 0.38 g of ethylene glycol and 0.12 g of TWEEN 80 and gently hand mixed for approximately five minutes, until all the carbon black is involved. Using 15 wt % of carbon black may achieve best conductivity of the conductive polymer. Using between 12-15 w % carbon black achieved good conductivity of the conductive polymer. That is 0.74 g of carbon black may be added to the above mixture. Alternatively, when adding 12w % of carbon black, 0.59 g (12 wt %) of carbon black may be added to the same amounts of other components.

TWEEN 80 is a non-ionic surfactant that improves miscibility of carbon black and PDMS and thus improves the mechanical properties of the conductive polymer. That is, TWEEN 80 acts as a ‘softener’, and depending on the amount of TWEEN 80, this results in spongier or more solid electrodes.

Ethylene Glycol is a polar organic compound that works as a secondary dopant and thus improves the electrical properties of the conductive polymer.

Next, a curing agent may be added to the mixture at step S102. Using the quantities specified above, after obtaining a uniform ‘paste’, add 0.07 g of PDMS part B to the mixture and mix again for five minutes. PDMS part B is the curing agent for PDMS and is added only at the end to avoid the mixture curing during the fabrication process, ensuring a better uniformity of the final paste.

The silicone polymer may be polydimethylsiloxane which is created when PDMS Part A (silicone monomer base) and Part B (curing agent) react and cure. When heated, PDMS Part A and Part B crosslink, forming a network structure of chemical bonds.

Optionally, as shown in FIG. 3, glycerol may be added at this stage (step S104). Adding glycerol as a further additive may improve the adhesive properties of the conductive polymer electrode. For example, 0.55 g of glycerol may be added to the mixture with quantities specified above at this stage if using 14-15 wt % of carbon black or 1 g (17 wt %) of glycerol if using 12-14 wt % of carbon black. Alternatively, when adding glycerol, 0.66 g may be added to the composition, with the above specified quantities of the other components.

Thus, to summarize, the following components may be used to obtain the example electrically-conductive polymer electrode composition described above:
Carbon black (C), C-Nergy Super C65, ≥99.975, PI-KEM
Ethylene Glycol—RPE (CH2OHCH2OH), 99.5% (GLC), Carlo Erba Reagents
TWEEN® 80 viscous liquid, Polyethylene glycol sorbitan monooleate, Sigma-AldrichPolydimethylsiloxane ((C₂H₆OSi)ₙ), Sylgard 184 Silicone Elastomer Kit, Dow:
Part A—Sylgard 184 Silicone Elastomer Base:
Dimethyl siloxane, dimethylivinyl terminated—68083-19-2
Dimethylvinylated and trimethylated silica—68988-89-6
Tetra (trimethoxysiloxy) silane—3555-47-3
Ethyl benzene—100-41-4
Part B—Sylgard 184 Silicone Elastomer Curing Agent:
Dimethyl, methylhydrogen siloxane—68037-59-2
Dimethyl siloxane, dimethylvinyl terminated—68083-19-2
Dimethylvinylated and trimethylated silica—68988-89-6
Tetramethyl tetravinyl cyclotetra siloxane—2554-06-5
Ethyl benzene—100-41-4
Glycerol (HOCH2CH(OH)CH2OH), BioXtra, >=99% (GC), Sigma Aldrich

The components of the conductive polymer may be present in the conductive polymer in the following proportions:

| | | w/w | |
|---|---|---|---|
| Component | Mass (g) | (solute/solution) % | Range± (g) |
| PDMS part A (base) | 3.59 | 72.41 | ±0.3 |
| PDMS part B | 0.07 | 1.42 | ±0.04 |
| (curing agent) | | | |
| Carbon black | 0.80 | 16.09 | ±0.07 |
| Ethylene Glycol | 0.38 | 7.57 | ±0.09 |
| Tween 80 | 0.12 | 2.51 | ±0.04 |

The above amounts refer to both the cured and uncured conductive polymer. Merely for illustrative purposes, the above table includes w %, as well as an example of a mixed composition given in units of mass (g). Different proportions of the constituent parts of the conductive polymer composition are possible. The conductive polymer composition may comprise at least 5 wt % particulate carbon. The conductive polymer composition may comprise no more than 20 wt % particulate carbon. The conductive polymer composition may comprise at least 9w % particulate carbon. The conductive polymer composition may comprise no more than 16w % particulate carbon. Particulate carbon may mean carbon black, as described above. The conductive polymer composition may comprise 14.9 w % particulate carbon. The lower limit of carbon black is dictated by conductivity requirements, whereas the higher limit is dictated by mechanical properties of the silicone polymer as well as the curing process. Adding more than 20 wt %, or in some cases more than 16 wt %, of carbon black may result in less favorable mechanical properties of the cured conductive polymer and higher skin impedance. However, in order to reach conductivity that is as high as possible, it may be advantageous to include enough carbon black as possible, without affecting the mechanical properties of the conductive polymer.

The conductive polymer composition may comprise 10 w % Triton X-100. The conductive polymer composition may comprise 7w % ethylene glycol. Alternatively, the conductive polymer composition may comprise 2.5 wt. % Tween 80. The conductive polymer composition may comprise 7.6 w % ethylene glycol. The resulting conductive polymer composition may have an electrical skin impedance of less than 2000 kΩ. The electrical skin impedance of the conductive polymer may be between 80 kΩ and 1000 kΩ.

Returning to FIG. 3, the method may comprise patterning a surface of the film of conductive polymer composition prior to curing (step S106). Preferably, this step is performed after the film of the conductive polymer composition has been applied to the at least one electrode pad 104 of the F-PCB. Thus, manufacturing a conductive polymer electrode may further comprise patterning a surface of the uncured conductive polymer to obtain an increased surface area of the cured conductive polymer/dry electrode 204.

Curing the conductive polymer mixture at step S108 may comprise at least one thermal curation step at a predetermined curation temperature. Controlling a temperature of a thermal curation step ensures that the curation process is controlled and therefore, along with controlling a ratio of a curing agent in the conductive polymer, allows control over the ratio of cured to uncured polymer chains in the cured conductive polymer. Controlling a temperature of a thermal curation step ensures that the curation process is controlled and therefore allows control over the ratio of cured to uncured polymer chains in the cured conductive polymer. In turn, control over the ratio of cured to uncured polymer chains means that the properties of the conductive polymer can be controlled. For example, a larger amount of cured chains results in a more solid conductive polymer, whereas less cured chains result in a softer conductive polymer. These are important properties, as the softness of the conductive polymer and other surface properties of the conductive polymer determine how well the conductive polymer conforms to a user's skin and thus how well the conductive polymer works as an interface between the electrode pad and the user's skin.

Thus, the sensor, with the at least one dry electrode, may be placed in an oven at step S108. The temperature of the oven may be in a range from room temperature to 110° C. The oven may be programmed to gradually increase the temperature from room temperature to a predetermined temperature of 100° C. over a predetermined time period of two hours. Next, the temperature may be in a range from 100° C. to 110° C. The oven may be programmed to increase the temperature from 100° C. to 110° C. over a two-hour period. That is, there may be a further increase in temperature of 5° C. per hour. The second rise in temperature is a slow rise in temperature, to avoid any cracking or other temperature-related defects on the conductive polymer electrodes. Finally, the temperature may decrease from 110° C. to room temperature. Thus, during this final step, the temperature may be lowered from 110° C. to room temperature over a 2.5 hour period.

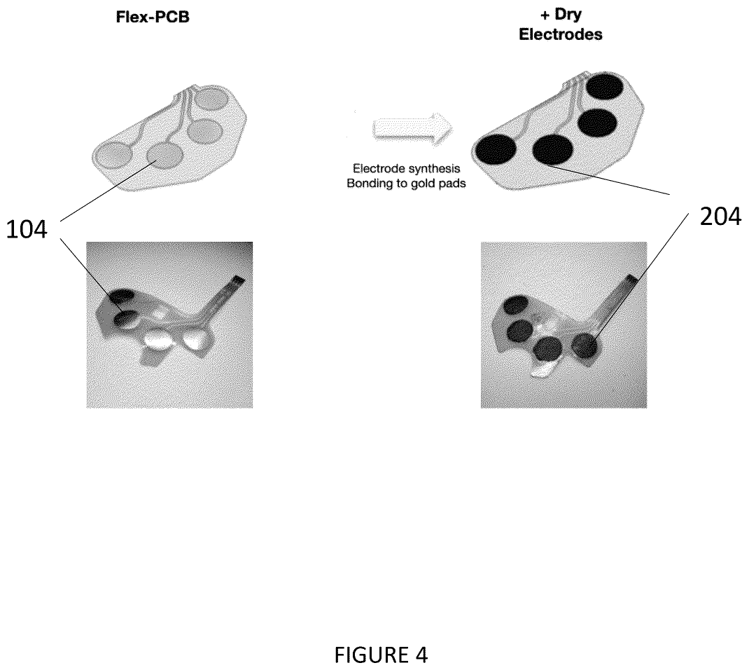

FIG. 4 is a diagram showing an overview of the overall process for coating the F-PCB of the sensor with the conductive polymer electrode composition. As shown in FIG. 4, and explained above, the present techniques provide a wearable sensor for monitoring physiological and/or brain signals, the sensor comprising: at least one electrode pad 104 for monitoring physiological and/or brain signals, wherein the at least one electrode pad 104 is at least partially coated in a coating formed of the conductive polymer composition described herein; and circuitry coupled to the at least one electrode pad. The conductive polymer composition coating may form a dry electrode 204 over each electrode pad 104, as shown in FIG. 4.

The coating may comprise patterning, as described above with reference to FIG. 2, to obtain an increased surface area of the conductive polymer. This may improve the ability for the coating (i.e. dry electrode) to contact the skin and sense signals.

The at least one electrode pad (and the coating) and the circuitry may be provided on a thin flexible substrate, and the circuitry may also be flexible. The substrate may have a thickness in a range from 0.04 to 0.3 mm. For example, the substrate may have a thickness of 0.08 mm. The substrate may be made from flexible materials, such as, for example, a polymer and/or polyimide layer and/or conductive material with a thickness in a range from 10 to 25 μm, for example, copper with a thickness of 18 μm. At least part of the substrate may have an immersion gold surface finish. A thin flexible substrate and flexible circuitry mean that the sensor adapts to a user's movement—the sensor moves along with the user's skin, even when the user is moving. Advantageously, this means that contact between a conductive polymer electrode interface and the user's skin is always maintained. Additionally, with the circuitry being flexible and provided on the flexible substrate, the circuitry will also move along with any movements of the user.

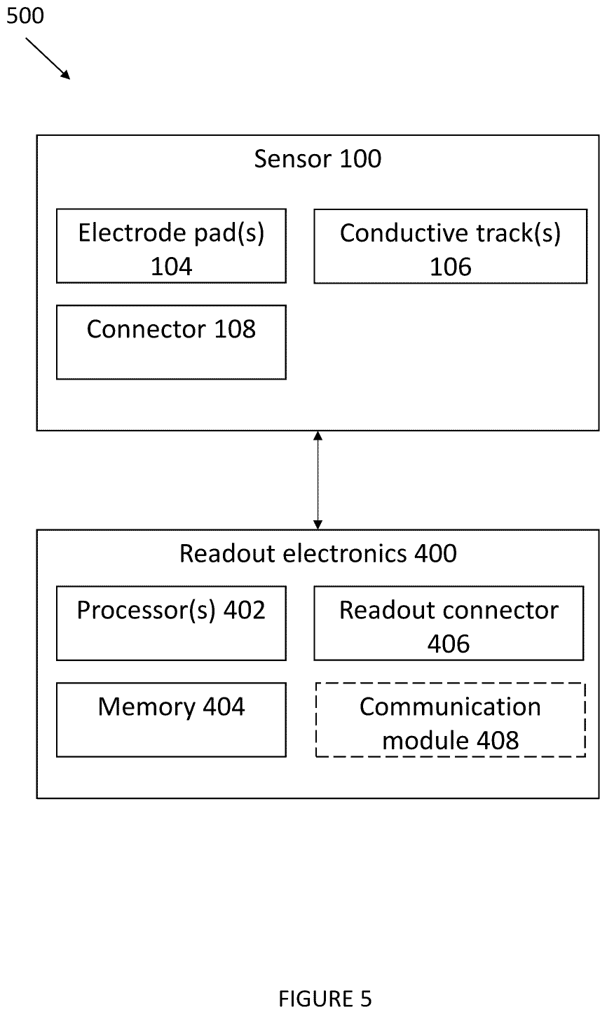

FIG. 5 is a block diagram of a system comprising the sensor as described above and a readout electronics device. The system 500 may comprise the sensor 100 as described above and a readout electronics 400 device. The sensor 100 comprises at least one electrode pad 104. The at least one electrode pad 104 may be used for EEG, ECG and/or EMG measurements (which are the measurements most susceptible to motion-induced noise) or measurement of any other surface electrophysiological signal from the body. When the sensor is provided on a user's head, the at least one electrode pad may sense brain signals. In this case, the at least one electrode pad may be able to provide (directly or indirectly) information on the cognition, emotional state, or disease indicators. The sensor may further comprise at least one conductive track 106. The at least one conductive track 106 may transmit measurements from the at least one electrode pad to a connector 108. The sensor 100 may also comprise the connector 108 deposited onto the PCB 102. The connector may be used to connect the sensor 100 to an apparatus and/or readout electronics for receiving a physiological signal that is measured by the sensor 100.

The system may further comprise readout electronics 400. The readout electronics may be connected via a readout connector 406 to the sensor's connector 108. The readout electronics may further comprise at least one processor 402 and memory 404. The processor and memory may be used to analyze physiological and/or brain signals measured by the sensor. Optionally, the readout electronics may also comprise a communication module 408 for transmitting a received physiological and/or brain signal to an external electronic device. Thus, the readout electronics may wirelessly transmit data to a mobile, desktop or cloud computing platform for further data processing and extraction of insights relating to a cognitive and physiological state of a user.

The readout electronics 400 may comprise: an analog front-end (which comprises an amplifier and a digitizer), a micro-controller unit, a wireless communication module (e.g. cellular, WiFi, Bluetooth, etc.) and other passive circuit elements required to make these components work.

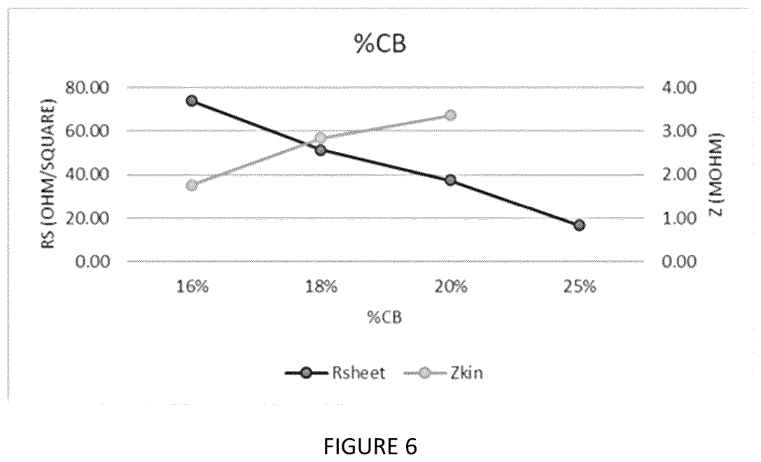

The appropriate range of carbon black was found based on electrical and mechanical electrode properties, such as skin impedance values and conductivity, as well as mechanical strength and wear resistance. For instance, while adding carbon can increase conductivity, it may reduce skin impedance and mechanical properties. FIG. 6 is a line graph showing the relationship between the concentration of carbon black by weight percent and the resistivity (blue, or dark shade, line); and the relationship between the concentration of carbon black by weight percent and skin impedance (yellow, or light shade, line). The x-axis represents the concentration of carbon black by weight (wt %), the left-hand y-axis represents resistivity of the electrode, the right-hand y-axis represents the skin impedance of the electrode. The data shown in FIG. 6 demonstrates that as the concentration of carbon black increases, so does the skin impedance of the electrode. FIG. 6 also shows that as the concentration of carbon black increases, the resistivity of the electrode decreases. Sufficient percolation of the carbon black was found to occur at a concentration above about 5 wt %. On the other hand, skin impedance and mechanical strength are limiting-factors at concentrations above about 20 wt % carbon black, despite a conductivity increase. The use of ≤20 wt % carbon black, by weight of the electrically-conductive polymer composition, achieves electrodes that meet electrophysiological recording needs.

As shown in FIG. 6, lower values for skin impedance (Z) and higher mechanical performance was achieved with smaller conductivity/higher resistivity (Rs) values. Lower impedance values during electrophysiological recording enable lower noise amount, which is crucial for EEG (small signal).

Those skilled in the art will appreciate that while the foregoing has described what is considered to be the best mode and where appropriate other modes of performing present techniques, the present techniques should not be limited to the specific configurations and methods disclosed in this description of the preferred embodiment. Those skilled in the art will recognize that present techniques have a broad range of applications, and that the embodiments may take a wide range of modifications without departing from any inventive concept as defined in the appended claims.

The invention may be defined in accordance with accordance with the following numbered aspects:

1. A dry electrode for a wearable sensor, the dry electrode being formed from an electrically-conductive polymer composition, the electrically-conductive polymer composition comprising: an electrically-conductive material comprising particulate carbon; at least one additive; and a silicone polymer; wherein the particulate carbon is present in a range from 5 wt. % to 20 wt. % by weight of the electrically-conductive polymer composition.

2. The dry electrode according to aspect 1 wherein the silicone polymer is polydimethylsiloxane.

3. The dry electrode according to any of aspects 1 or 2 wherein the particulate carbon has an average particle size ranging from 5 to 100 nm.

4. The dry electrode according to any of aspects 1 to 3 wherein the particulate carbon is present in a range from 14 wt. % to 17 wt. %, by weight of the electrically-conductive polymer composition.

5. The dry electrode according to any of aspects 1 to 4 wherein the at least one additive comprises a surfactant, for example Triton X-100 and/or Tween 80.

6. The dry electrode according to aspect 5 wherein the surfactant is Triton X-100, and the Triton X-100 is present in a range from 1wt. % to 30 wt. %, preferably in a range from 1wt. % to 20 wt. %, more preferably in a range from 5 wt. % to 15 wt. %, for example about 10wt. % such as 10wt. %, by weight of the conductive polymer composition.

7. The dry electrode according to aspect 5 wherein the surfactant is Tween 80, and the Tween 80 is present in a range from 1 wt. % to 20 wt. %, preferably in a range from 1 wt. % to 10 wt. %, more preferably in a range from 1 wt. % to 5 wt. %, for example about 2.5 wt. % such as 2.51 wt. %, by weight of the conductive polymer composition.

8. The dry electrode according to any preceding aspect wherein the at least one additive comprises a first additive and a second additive.

9. The dry electrode according to aspect 8 wherein the second additive is ethylene glycol.

10. The dry electrode according to aspect 9 wherein the ethylene glycol is in a range from 1wt. % to 30 wt. %, preferably in a range from 1wt. % to 20 wt. %, more preferably in a range from 3 wt. % to 15 wt. %, most preferably in a range from 6 wt. % to 8 wt. %, for example about 7 wt. %, such as 7 wt. %, by weight of the conductive polymer composition.

11. The dry electrode to any preceding aspect wherein at 30 Hz, an electrical skin impedance of the conductive polymer is in a range from 100 kΩ to 2000 kΩ, preferably in a range from 100 kΩ to 1000 kΩ, more preferably in a range from 50 kΩ to 500 kΩ, most preferably in a range from 10 kΩ to 200 kΩ.

12. A dry electrode according to any preceding aspects, wherein the electrically-conductive polymer composition is derived from an electrically-conductive polymer mixture comprising: an electrically-conductive material comprising particulate carbon; at least one additive; a silicone polymer; and a curing agent, wherein the particulate carbon is present in a range from 5 wt. % to 20 wt. % by weight of the electrically-conductive polymer mixture.

13. The dry electrode according to aspect 12 wherein the electrically-conductive material is particulate carbon, and/or the at least one additive is a surfactant, such as Triton X-100 and/or Tween 80, and/or the silicone polymer is uncured polydimethylsiloxane, and/or the curing agent is a curing agent for polydimethylsiloxane.

14. A method for manufacturing a dry electrode for a wearable sensor, the method comprising: obtaining an electrically-conductive polymer mixture by mixing together an electrically-conductive material comprising particulate carbon, at least one additive, a silicone polymer, and optionally a curing agent, wherein the particulate carbon is present in a range from 5 wt. % to 20 wt. % by weight of the electrically-conductive polymer mixture; and curing the electrically-conductive polymer mixture to obtain a cured electrically-conductive polymer composition.

15. A wearable sensor for monitoring physiological and/or brain signals, the sensor comprising: at least one electrode pad for monitoring physiological and/or brain signals, wherein a skin-facing surface of the at least one electrode pad is at least partially coated in a coating formed of the electrically-conductive polymer composition according to any of aspects 1 to 13.

16. The wearable sensor according to aspect 15 further comprising circuitry electrically coupled to the at least one electrode pad.

17. The wearable sensor according to any of aspects 15 or 16 wherein the coating comprises patterning for increasing a surface area of the coating.

18. The wearable sensor according to any of aspects 15, 16 or 17 wherein the at least one electrode pad and the circuitry are provided on a thin flexible substrate, and wherein the circuitry is flexible.

19. A method for manufacturing at least one electrode of a wearable sensor for monitoring physiological and/or brain signals, the wearable sensor having at least one electrode pad, the method comprising: coating at least part of the at least one electrode pad in an electrically-conductive polymer mixture, the mixture comprising an electrically-conductive material comprising particulate carbon, at least one additive, a silicone polymer, and optionally a curing agent, wherein the particulate carbon is present in a range from 5 wt. % to 20 wt. % by weight of the electrically-conductive polymer mixture; and curing the electrically-conductive polymer mixture to obtain a cured electrically-conductive polymer coating on the at least one electrode pad.

20. The method according to aspect 19 further comprising: patterning a surface of the electrically-conductive polymer, prior to curing, to increase a surface area of the coating.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

1. A dry electrode for a wearable sensor, the dry electrode being formed from an electrically-conductive polymer composition, the electrically-conductive polymer composition comprising:
an electrically-conductive material comprising particulate carbon;
at least one additive; and
a silicone polymer;
wherein the particulate carbon is present in a range from 5 wt. % to 20 wt. % by weight of the electrically-conductive polymer composition.

2. The dry electrode as claimed in claim 1 wherein the silicone polymer is polydimethylsiloxane.

3. The dry electrode as claimed in claim 1 wherein the particulate carbon has an average particle size ranging from 5 to 100 nm.

4. The dry electrode as claimed in claim 1 wherein the particulate carbon is present in a range from 14 wt. % to 17 wt. % by weight of the electrically-conductive polymer composition.

5. The dry electrode as claimed in claim 1 wherein the at least one additive comprises a surfactant.

6. The dry electrode as claimed in claim 5 wherein the surfactant is Triton X-100, and the Triton X-100 is present in a range from 1wt. % to 30 wt. % by weight of the conductive polymer composition.

7. The dry electrode as claimed in claim 5 wherein the surfactant is Tween 80, and the Tween 80 is present in a range from 1 wt. % to 20 wt. % by weight of the electrically-conductive polymer composition.

8. The dry electrode as claimed in claim 1 wherein the at least one additive comprises a first additive and a second additive.

9. The dry electrode as claimed in claim 8 wherein the second additive is ethylene glycol.

10. The dry electrode as claimed in claim 9 wherein the ethylene glycol is in a range from 1wt. % to 30 wt. % by weight of the conductive polymer composition.

11. The dry electrode as claimed in claim 1 wherein at 30 Hz, an electrical skin impedance of the conductive polymer is in a range from 100 kΩ to 2000 kΩ.

12. A dry electrode according to claim 1, wherein the electrically-conductive polymer composition is derived from an electrically-conductive polymer mixture comprising:
an electrically-conductive material comprising particulate carbon;
at least one additive;
a silicone polymer; and
a curing agent,
wherein the particulate carbon is present in a range from 5 wt. % to 20 wt. % by weight of the electrically-conductive polymer mixture.

13. The dry electrode as claimed in claim 12 wherein
the electrically-conductive material is particulate carbon, or
the at least one additive is a surfactant, or
the silicone polymer is uncured polydimethylsiloxane, or
the curing agent is a curing agent for polydimethylsiloxane, or
any combination thereof.

14. The dry electrode according to claim 1, wherein the dry electrode is manufactured by a method comprising:
obtaining an electrically-conductive polymer mixture by mixing together an electrically-conductive material comprising particulate carbon, at least one additive, a silicone polymer, and optionally a curing agent, wherein the particulate carbon is present in a range from 5 wt. % to 20 wt. % by weight of the electrically-conductive polymer mixture; and
curing the electrically-conductive polymer mixture to obtain a cured electrically-conductive polymer composition.

15. A wearable sensor for monitoring physiological, or brain signals, or both, the sensor comprising:
at least one electrode pad for monitoring physiological, or brain signals, or both, wherein a skin-facing surface of the at least one electrode pad is at least partially coated in a coating formed of the electrically-conductive polymer composition as claimed in claim 1.

16. The wearable sensor as claimed in claim 15 further comprising circuitry electrically coupled to the at least one electrode pad.

17. The wearable sensor as claimed in claim 15 wherein the coating comprises patterning for increasing a surface area of the coating.

18. The wearable sensor as claimed in claim 16 wherein the at least one electrode pad and the circuitry are provided on a thin flexible substrate, and wherein the circuitry is flexible.

19. A method for manufacturing at least one electrode of a wearable sensor for monitoring physiological, or brain signals, or both, the wearable sensor having at least one electrode pad, the method comprising:
coating at least part of the at least one electrode pad in an electrically-conductive polymer mixture, the mixture comprising an electrically-conductive material comprising particulate carbon, at least one additive, a silicone polymer, and optionally a curing agent, wherein the particulate carbon is present in a range from 5 wt. % to 20 wt. % by weight of the electrically-conductive polymer mixture; and
curing the electrically-conductive polymer mixture to obtain a cured electrically-conductive polymer coating on the at least one electrode pad.

20. The method as claimed in claim 19 further comprising: patterning a surface of the electrically-conductive polymer mixture, prior to curing, to increase a surface area of the coating.
